(19) United States
(12) Patent Application Publication
(10) Pub. No.: US 2012/0052011 A1
(43) Pub. Date: Mar. 1, 2012

(54) COMPOSITION AND A METHOD FOR PRODUCING CONTRAST AGENT USING THE COMPOSITION

(75) Inventors: Tatsuki Fukui, Yokohama-shi (JP); Satoshi Yuasa, Yokohama-shi (JP); Kouichi Kato, Yokohama-shi (JP)

(73) Assignee: CANON KABUSHIKI KAISHA, Tokyo (JP)

(21) Appl. No.: 13/205,246

(22) Filed: Aug. 8, 2011

(30) Foreign Application Priority Data
Aug. 24, 2010 (JP) 2010-187500

(51) Int. Cl. A61K 49/00; A61P 43/00

(52) U.S. Cl. 424/9.1

(57) ABSTRACT

A composition including a hydrophilic dye having a sulfonate group and a hydrophobic solvent, wherein the composition includes at least one of a nicotinic acid derivative and a tiamine derivative, can form densely accumulated particles and the like since the composition includes a hydrophilic dye that is likely to dissolve in a hydrophobic solvent.

### BACKGROUND OF THE INVENTION

1. Field of the Invention

The present invention relates to a composition including a hydrophilic dye, which is improved in solubility in a hydrophobic solvent, and a method for producing a contrast agent using the composition.

2. Description of the Related Art

As an imaging method capable of performing noninvasive diagnosis, a fluorescence method and photoacoustic tomography attract attention in recent years. Photoacoustic tomography is a method of detecting acoustic wave that is generated by volume expansion occurred by heat released from a molecule absorbing optical energy. In the fluorescence method and photoacoustic tomography, a dye can be used as a contrast agent, in order to amplify the signal intensity at the observation subject site. In this case, it is desirable to use a hydrophilic dye that is easy to be discharged from the living body to the outside of the body and has higher safety when used on the clinical examination. Indocyanine green (Indocyanine Green, hereinafter, may be also abbreviated as ICG), that is a near-infrared fluorescent dye, is an approved pharmaceutical and used for a liver function test, a circulatory function test, and diagnosis for the identification of sentinel lymph nodes in breast cancer. Namely, ICG, whose safety in administration to the human body is confirmed, is one hydrophilic dye expected to be used as a contrast agent of photoacoustic tomography.

In order to effectively amplify the signal intensity when used in photoacoustic tomography, it is desirable to accumulate a dye on particles, micelle, polymer micelles and liposome (collectively referred to as particles and the like). It is because accumulation increases the dye density and increases the efficiency of irradiation energy absorption, and also particularly when the dye is a fluorescent dye, accumulation causes quenching to prevent irradiation energy from being used for fluorescence emission and convert irradiation energy to more thermal energy.

Colloids and Surfaces B: Biointerfaces 75 (2010) 260-267 reports results of accumulating ICG in the polymer nanoparticles by a nanoemulsion method, by dissolving ICG in methanol, that is a hydrophilic solvent, and mixing with dichloromethane, that is a hydrophobic solvent.

On the other hand, Journal of Biomedical Optics 131, 014025, 2008 reports results of accumulating ICG in the polymer nanomicelles by a Nanoemulsion method, by preparing a tetrabutylammonium salt of ICG and dissolving in chloroform, that is a hydrophobic solvent.

### SUMMARY OF THE INVENTION

A hydrophilic dye is hardly soluble in a hydrophobic solvent. Therefore, in Colloids and Surfaces B: Biointerfaces 75 (2010) 260-267, ICG is dissolved in methanol, that is a hydrophilic solvent, not dichloromethane, and used by mixing with dichloromethane, to prepare particles.

However, in this method, the hydrophilic dye diffuses out of the particles in the step of dispersing in water an organic solvent in which a material including a hydrophilic dye at the preparation of polymer particles, and thus it is difficult to accumulate the dye into the particles in high concentration.

In addition, in Journal of Biomedical Optics 131, 014025, 2008, since ICG is not soluble in chloroform that is a hydrophobic solvent, an ICG tetrabutylammonium salt is prepared using a tetrabutylammonium salt, and dissolved in chloroform, to prepare particles.

However, in this method, the amount of a salt including a hydrophilic dye and tetrabutylammonium dissolved in chloroform is small, and thus, it is difficult to accumulate the hydrophilic dye into the micelles in high concentration.

The present inventors have studied to provide by an emulsion method a composition for preparing particles and the like in which a hydrophilic dye is densely accumulated. The emulsion method herein refers to a method of forming particles by mixing a hydrophilic solvent and a hydrophobic solvent to make the liquid-liquid dispersion state.

The present invention relates to a composition including a hydrophilic dye having a sulfonate group and a hydrophobic solvent, wherein the composition includes at least one of a nicotinic acid derivative and a tiamine derivative.

The present invention enables the dissolution of a hydrophilic dye in a hydrophobic solvent and enables the accumulation of the hydrophilic dye on particles and the like in high concentration. Since the solubility of a hydrophilic dye such as ICG in a hydrophobic solvent is improved, the application range of the hydrophilic dye can be broadened.

Further features of the present invention will become apparent from the following description of exemplary embodiments with reference to the attached drawings.

### BRIEF DESCRIPTION OF THE DRAWINGS

FIG. 1 is a result of evaluating the solubility of a hydrophilic dye-containing composition in chloroform in Examples A-1 and A-2 and Comparative Examples A-0, A-3 and A-4.

FIG. 2 is results of evaluating the solubility of a hydrophilic dye-containing composition in dichloromethane in Examples B-1 and B-2 and Comparative Examples B-0 and B-5 to B-7.

FIG. 3 is results of evaluating the amount of ICG transferred in a dichloromethane solution of a hydrophilic dye-containing composition to water in Example WB.

FIG. 4 is a chart for describing one example of the method for producing a contrast agent for optical imaging according to the present embodiment.

### DESCRIPTION OF THE EMBODIMENTS

Preferred embodiments of the present invention will now be described in detail in accordance with the accompanying drawings.

One embodiment of the present invention relates to a composition including a hydrophilic dye having a sulfonate group, a hydrophobic solvent, and at least one of a nicotinic acid derivative and a tiamine derivative.

In one embodiment of the present invention, the solubility of the hydrophilic dye having a sulfonate group in the hydrophobic solvent is increased by including a nicotinic acid derivative and/or a tiamine derivative. When this composition is mixed with water to form particles by an emulsion method, the amount of the dye having the sulfonate group eluted from the particles into water is suppressed. Therefore, when the composition according to the present embodiment is used, particles with less elution of the hydrophilic dye having a sulfonate group into water can be prepared, and a contrast agent that can be effectively used for a fluorescence method and photoacoustic tomography can be prepared. The composition according to the present embodiment contains 0.01-200 mg/ml, preferably 0.1-100 mg/ml of the hydrophilic dye having the sulfonate group in the hydrophobic solvent.

The composition according to the present embodiment can be a composition used for manufacturing a contrast agent for photoimaging. The composition according to the present embodiment can further be a composition used for manufacturing a contrast agent for photoacoustic imaging.

Incidentally, it is considered that, the reason why the solubility of the hydrophilic dye having a sulfonate group in the hydrophobic solvent is increased by adding a nicotinic acid derivative and/or a tiamine derivative to a hydrophilic dye having a sulfonate group is as follows. Namely, it is probably because a nicotinic acid derivative and/or a tiamine derivative assembles to a hydrophilic group of the hydrophilic dye having a sulfonate group, and the hydrophilic dye having a sulfonate group is likely to dissolve in the hydrophobic solvent. Particularly, the contribution of a nicotinic acid backbone having a pyridine ring in a nicotinic acid derivative, a pyrimidine ring in a tiamine derivative, or an amino group is considered. Incidentally, the nicotinic acid backbone having a pyridine ring specifically in the present invention means a structure obtained by removing NH₂ from nicotinamide.

(Hydrophilic Dye Having Sulfonate Group)

When used in the living body, it is safe and desirable that the hydrophilic dye having sulfonate group is a hydrophilic dye which is likely to be discharged from the living body. In addition, in the composition according to the present embodiment, since a nicotinic acid derivative and/or a tiamine derivative is considered to assemble to a sulfonate group and exhibits an effect, a hydrophilic dye having a sulfonate group can be especially preferable. In addition, as the hydrophilic dye, one having an absorption at a wavelength of 600 nanometer or more and 1300 nanometer or less, that has less effects of absorption and diffusion of light in the living body and is called the “biological window” can be preferable. Hereinafter, the hydrophilic dye having sulfonate group may simply be referred to as a hydrophilic dye.

Examples of the hydrophilic dye can include azine dyes, acridine dyes, triphenylmethane dyes, xanthene dyes, porphyrin dyes, cyanine dyes, phthalocyanine dyes, styryl dyes, pylyrium dyes, azo dyes, quinone dyes, tetracycline dyes, flavone dyes, polyene dyes, BODIPY (registered trademark) dyes, and indigoid dyes.

Examples of the cyanine dyes can include indocyanine green (ICG), Alexa Fluor (registered trademark) dyes (manufactured by Invitrogen), Cy (registered trademark) dyes (manufactured by GE Healthcare Bioscience), IR-783, IR-806 and IR-820 (manufactured by Sigma-Aldrich Japan), IRDye 800CW and IRDye 800RS (registered trademark) (manufactured by LI-COR), and ADS780WS, ADS795WS, ADS830WS and ADS832WS (manufactured by American Dye Source, Inc.).

Incidentally, in the present embodiment, ICG (indocyanine green) is a structure illustrated by the following Chemical Formula 1, wherein the counterion may not be Na⁺, and for example, may be H⁺ or K⁺.

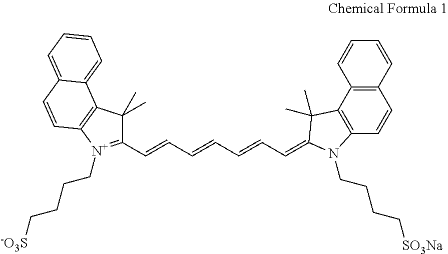

Incidentally, the IR-820 is represented by the following chemical formula 2.

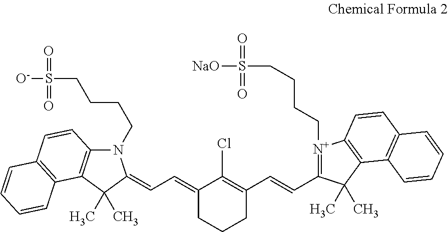

Examples of the indigoid dyes can include indigo carmine.

These hydrophilic dyes may be used alone or may be optionally mixed and used.

In addition, in the present embodiment, hydrophilic dye such as Patent Blue can be also used.

(Nicotinic Acid Derivative)

The nicotinic acid derivative in the present embodiment is not particularly limited so long as having a nicotinic acid backbone, but is preferably represented by the following formula (I).

A-Z (I)

(in the formula (I), A represents one of formulae (a1), (a2) and (a3) below, and

Z represents one of formulae (z1), (z2) and (z3) below)

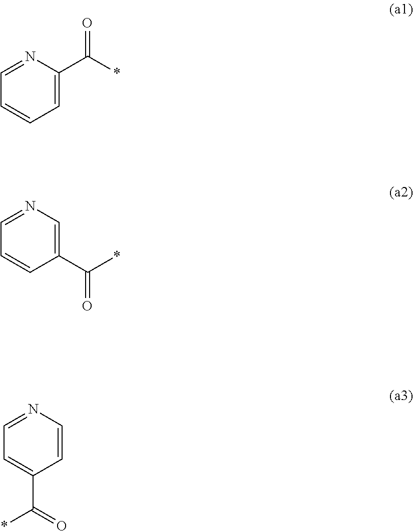

(in the formulae (a1), (a2) and (a3), * represents a bonding hand bound to Z of formula (I))

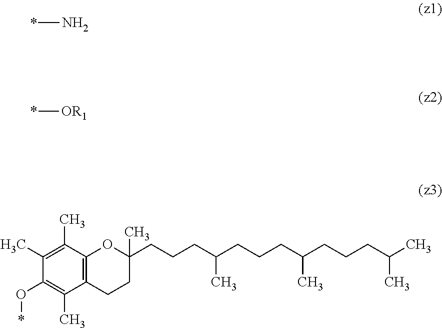

(in the formulae (z1), (z2) and (z3), * represents a bonding hand bound to A of formula (I),

in the formula (z2), R₁ is one of a hydrogen atom, a substituted or unsubstituted alkyl group having 1 to 10 carbon atoms and a benzyl group, and

the substituent is one of a halogen atom, an alkyl group having 1 to 5 carbon atoms, a hydroxyl group and an amino acid).

When the nicotinic acid derivative in the present embodiment is represented by the above formula (I), hydrogen ions attracted to the nitrogen atom of the pyridine ring counteracts against the sulfonate group of the hydrophilic dye having sulfonate group, to thereby facilitate the solubility of the hydrophilic dye in the hydrophobic solvent. Therefore, irrespective of whether the —COR₁ is in any of the ortho, meta and para positions, the hydrophilic dye having the nicotinic acid derivative represented by the above formula (I) and the sulfonate group, compared to a single body of the hydrophilic dye, has a high solubility to the hydrophobic solvent.

The nicotinic acid derivative in the present embodiment includes nicotinamide, benzyl nicotinate, nicotinic acid, methyl nicotinate, ethyl nicotinate, ethyl isonicotinate and tocopherol nicotinate. Among these, the nicotinic acid derivative is preferably at least one of nicotinamide and benzyl nicotinate.

The nicotinic acid derivative may be used alone or may be optionally mixed and used.

(Tiamine Derivative)

The tiamine derivative in the present embodiment is not particularly limited, but is preferably represented by the following formula (II):

X—B—Y (II)

(in the formula (II), B represents the formula (b) below,

X represents one of formulae (x1), (x2), (x3) and (x4) below, and

Y represents one of formulae (y1) and (y2) below)

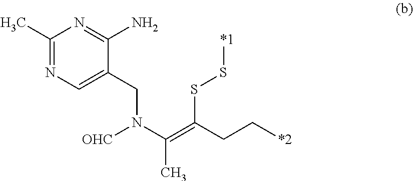

(in the formula (b), *1 represents a bonding hand bound to X of the formula (II), *2 represents a bonding hand bound to Y of the formula (II))

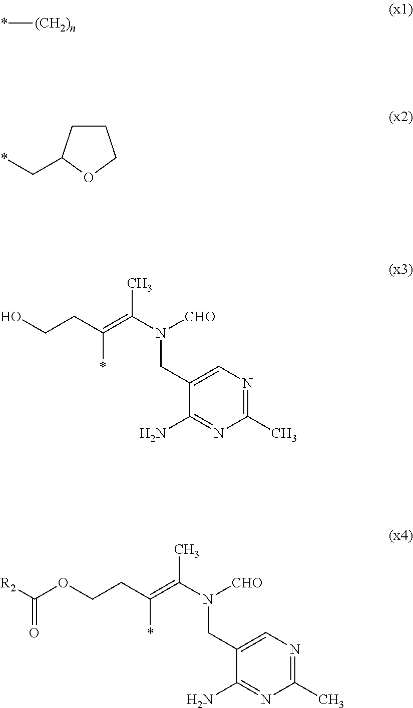

(in the formulae (x1), (x2), (x3) and (x4), * represents a bonding hand bound to B of formula (II),

in the formula (x1), n is an integer selected from 1 to 10, the formula (x1) may be substituted by a halogen atom, an alkyl group having 1 to 5 carbon atoms, a hydroxyl group and an amino acid,

in the formula (x4), R₂ is one of a substituted or unsubstituted alkyl group having 1 to 10 carbon atoms and a substituted or unsubstituted benzene)

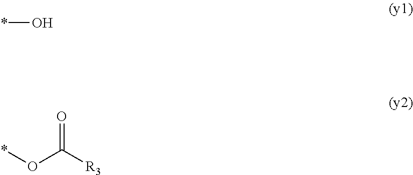

(in the formulae (y1) and (y2), * represents a bonding hand bound to B of formula (II),

in the formula (y2), R₃ is one of a substituted or unsubstituted alkyl group having 1 to 10 carbon atoms and a substituted or unsubstituted benzene).

The tiamine derivative includes tiamine disulfide, prosultiamine, fursultiamine, bisbentiamine and sulbutiamine. Among these, the nicotinic acid derivative is preferably at least one of fursultiamine, prosultiamine and tiamine disulfide.

The tiamine derivative may be used alone or may be optionally mixed and used.

In addition, nicotinic acid derivative and tiamine derivative may be optionally mixed and used.

(Hydrophobic Solvent)

The composition according to the present embodiment has an advantage in high solubility in a hydrophobic solvent. In the present embodiment, a hydrophobic solvent is a solvent that is not freely mixed with water. On the other hand, a solvent that is freely mixed with water is a hydrophilic solvent. “Freely mixed” herein means that, when 2 or more solvents are mixed, the solvents uniformly commingle without causing phase separation.

The hydrophobic solvent in the present embodiment includes hydrocarbons such as hexane, cyclohexane and heptane, ethers such as dimethylether and diethylether, halogenated hydrocarbons such as dichloromethane, chloroform, carbon tetrachloride, dichloroethane, and trichloroethane, aromatic hydrocarbons such as benzene and toluene, and esters such as ethyl acetate and butyl acetate. Among these, the hydrophobic solvent is preferably at least one of chloroform and dichloromethane.

The hydrophobic solvent may be used alone or may be optionally mixed and used.

(Preparation Method of Composition)

The composition according to the present embodiment is obtained by mixing a hydrophilic dye with a nicotinic acid derivative and/or tiamine derivative. A known method can be used for mixing, and the method is not particularly limited.

It can be preferable that the amount of the nicotinic acid derivative and/or tiamine derivative in the composition according to the present embodiment is in the range of 0.1 to 100.0-fold mol and preferably 1.0-fold to 20.0-fold mol based on the amount of the hydrophilic dye.

When the hydrophilic dye is mixed with the nicotinic acid derivative and/or tiamine derivative, a solvent can be used as necessary. In this time, as the solvent, hydrocarbons such as hexane, cyclohexane and heptane, ketones such as acetone and methyl ethyl ketone, ethers such as dimethylether, diethylether and tetrahydrofuran, halogenated hydrocarbons such as dichloromethane, chloroform, carbon tetrachloride, dichloroethane, and trichloroethane, aromatic hydrocarbons such as benzene and toluene, alcohols such as ethanol, methanol and isopropanol, aprotic polar solvents such as N,N-dimethylformamide and dimethylsulfoxide, and esters such as ethyl acetate and butyl acetate, pyridine derivatives, buffers such as a phosphoric acid buffer, and water can be used. The solvent is preferably a solvent in which the hydrophilic dye and the nicotinic acid derivative and/or tiamine derivative are soluble. The amount of the solvent used may be properly determined depending on the amount of the hydrophilic dye used, the amount of the nicotinic acid derivative and/or tiamine derivative used, and mixing conditions.

The mixing temperature is not particularly limited, and generally in the range of −30° C. to the boiling point of the used solvent when a solvent is used. However, it is desirable to properly carry out mixing at the optimal temperature according to the hydrophilic dye, the nicotinic acid derivative and/or tiamine derivative, and the solvent. The mixing time cannot be unconditionally determined, but is generally preferably in the range of 1 to 48 hours.

The solvent of the mixing solution can be removed by distillation in the usual manner or using column chromatography as necessary, or the composition may contain the solvent.

When mixing, the nicotinic acid derivative and/or tiamine derivative may be added after the hydrophilic dye is dispersed in the hydrophobic solvent, or the hydrophilic dye may be added after the nicotinic acid derivative and/or tiamine derivative is dispersed or dissolved in the hydrophobic solvent.

In addition, the composition according to the present embodiment may contain the solvent or may be dispersed or dissolved in the solvent, as necessary.

(Particle Formation)

In the composition according to the present embodiment, the amount of the hydrophilic dye eluted in water when mixed with water is small. Therefore, when particles containing ICG are formed by the step of forming O/W liquid-liquid dispersion state in water followed by the step of immobilizing an organic solvent phase, the efficiency of the hydrophilic dye can be increased. As the step of immobilizing an organic solvent, a conventionally known step can be used. As one example, to the composition according to the present embodiment are added one of a polymerizable monomer and a prepolymer soluble in the composition according to the present embodiment and a crosslinking agent, and the resulting product is made into liquid-liquid dispersion state in water, then the polymerizable material is subjected to a polymerization reaction, whereby an organic solvent phase can be immobilized and formed into particles. In order to progress a radical polymerization reaction, a low-temperature thermal polymerization initiator and a redox-active radical generator can be suitably used. As another example, a dicarboxylic acid chloride is added to the composition, and the mixture is made into liquid-liquid dispersion in an aqueous solution of diamine, whereby a polycondensed polymer membrane including the dicarboxylic acid and the diamine can be formed at the interface, and the organic solvent phase can be contained and immobilized. Furthermore, a surfactant can be used on particle formation. The particles formed thereby can be used as a contrast agent effectively used for a fluorescence method and photoacoustic tomography.

### <Method for Producing Contrast Agent for Optical Imaging>

The method for producing a contrast agent for optical imaging according to the present embodiment is characterized by including the steps of preparing a composition including a hydrophilic dye including a sulfonate group, a hydrophobic solvent, at least one of a nicotinic acid derivative and a tiamine derivative, and one of a polymerizable monomer and a prepolymer, mixing the composition with water, and polymerizing one of the polymerizable monomer and the prepolymer after the mixing step.

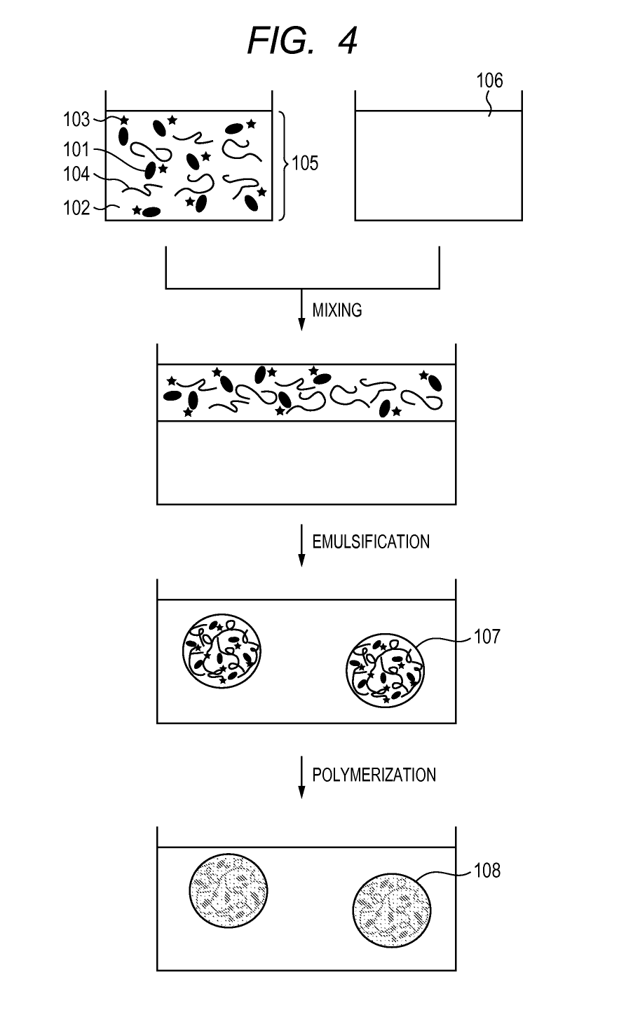

One example of the method for producing a contrast agent for optical imaging according to the present embodiment is described using FIG. 4.

First, a composition 105 including a hydrophilic dye 101 including a sulfonate group, a hydrophobic solvent 102, at least one of a nicotinic acid derivative and a tiamine derivative 103, and one of a polymerizable monomer and a prepolymer 104 is prepared. Subsequently, the composition 105 and water 106 are mixed. The liquid obtained by mixing is irradiated with ultrasound or the like, to give an emulsion. Dispersoid 107 of the emulsion obtained in this time has the composition 105. Among the dispersoid of the emulsion, the polymerizable monomer or the prepolymer is polymerized, whereby a contrast agent 108 for optical imaging can be obtained.

Incidentally, before polymerizing the polymerizable monomer or the prepolymer, it is preferable to remove the hydrophobic solvent from the dispersoid of the emulsion. The method of removing the hydrophobic solvent includes a method of reducing pressure.

<Contrast Agent for Optical Imaging>

The optical imaging in the present embodiment means that performing imaging by irradiation with light. In other words, to the hydrophilic dye including a sulfonate group of the contrast agent for optical imaging according to the present embodiment is irradiated with light, thereby emitting an acoustic wave, fluorescence and the like. Photoacoustic imaging can be performed by detecting the emitted acoustic wave, and fluorescent imaging can be performed by detecting the emitted fluorescence. Incidentally, the photoacoustic imaging is a concept including photoacoustic tomography (tomography).

The contrast agent according to the present embodiment may further include a dispersoid, for example, a physiological saline, distilled water for injection, a phosphate buffered saline (hereinafter, may be also abbreviated as PBS), and the like. In addition, the contrast agent for optical imaging according to the present embodiment may also include a pharmaceutically acceptable additive as necessary.

The contrast agent for optical imaging according to the present embodiment may be preliminarily dispersed in the dispersoid or may be made into a kit and dispersed in the dispersoid before administrating the agent to the living body, and used. As described above, the contrast agent for optical imaging according to the present embodiment can be used as a contrast agent for photoacoustic imaging and a contrast agent for fluorescent imaging.

The contrast agent for optical imaging according to the present embodiment can be accumulated more in a tumor site as compared to in a normal site in the living body by using Enhanced Permeability and Retention (EPR) effect when administrating to the living body. As a result, after administrating a complex to the living body, when the living body is irradiated with light to detect an acoustic wave and fluorescence from the living body, the acoustic wave and fluorescence emitted from the tumor site can be increased more than the acoustic wave and fluorescence emitted from the normal site. Therefore, the complex according to the present embodiment can be used as a contrast agent for optical imaging specifically detecting a tumor site.

(One of Polymerizable Monomer and Prepolymer)

In the present embodiment, the polymerizable monomer and the prepolymer are not particularly limited as long as it is soluble in the composition including a hydrophilic dye including a sulfonate group, a hydrophobic solvent, and at least one of a nicotinic acid derivative and a tiamine derivative. Here, the prepolymer is an intermediate product obtained by stopping one of the polymerization and condensation reaction of the polymerizable monomer at a certain point in the reaction and refers to a product at a stage before being a polymer.

Examples of the polymerizable monomer can include stylene, methacrylic acid, butyl methacrylate, a butyl methacrylate-methyl methacrylate copolymer, lactic acid, and glycolic acid.

(Polymerization Initiator)

As a polymerization initiator that polymerizes one of the polymerizable monomer and the prepolymer, conventionally known polymerization initiators can be used. Specific examples include azo initiators, peroxide initiators, redox initiators, atom transfer radical initiators, and nitroxide initiators. Specifically, it is preferable to use the azo initiators and peroxide initiators that come in many varieties and thus a suitable initiator can be selected based on the type of the monomer, and are easily available and cheap.

(Surfactant)

Examples of the surfactant can include polyoxyethylene alkyl ethers, alkyl sulfates, phospholipids, polyoxyethylene sorbitan fatty acid esters, polyvinyl alcohols and polyoxyethylene polyoxypropylene glycol.

The polyoxyethylene sorbitan fatty acid esters include Tween 20, Tween 40, Tween 60 and Tween 80.

The phospholipids can include phosphatidyl phospholipids including one of the functional groups of an amino acid, an NHS group, maleimide and a methoxy group and a PEG chain.

The phosphatidyl phospholipids can include 3-(N-succinimidyloxyglutaryl)aminopropyl, polyethyleneglycol-carbamyl distearoylphosphatidyl-ethanolamine(DSPE-PEG-NHS), N-(3-maleimide-1-oxopropyl)aminopropyl polyethyleneglycol-carbamyl distearoylphosphatidyl-ethanolamine(DSPE-PEG-MAL), N-(aminopropyl polyethyleneglycol)-carbamyl distearoylphosphatidyl-ethanolamine(DSPE-PEG-NH2), N-(Carbonyl-methoxypolyethyleneglycol 2000)-1,2-distearoyl-sn-glycero-3-phosphoethanolamine, sodium salt(SUNBRIGHT DSPE-020CN), and N-(Carbonyl-methoxypolyethyleneglycol 5000)-1,2-distearoyl-sn-glycero-3-phosphoethanolamine, sodium salt(SUNBRIGHT DSPE-050CN).

The polyoxyethylene polyoxypropylene glycol can include Pluronic (registered trademark) F68 and Pluronic (registered trademark) F127.

These surfactants may be used alone or may be optionally mixed and used.

(Polymer)

The polymer in the present invention can include homopolymers of hydroxy carboxylic acid having a carbon number of 6 or less or copolymers including two kinds thereof, polystylene and polymethacrylic acid.

In addition, derivatives of these polymers are usable.

The average molecular weight of these polymers is 2000 to 1000000, and the polymers with the average molecular weight of 10000 to 600000 can be preferably used.

These polymers may be used alone or may be optionally mixed and used.

### EXAMPLE

Hereinafter, the present invention is described according to examples, in order to further clarify the characteristics of the present invention. However, the present invention is not limited to these examples, and the materials and composition conditions can be freely changed in the range where the composition having similar functions and effects.

### Example A-1

11 mg of ICG (manufactured by Pharmaceutical and Medical Device Regulatory Science Society of Japan) was dissolved in 4 mL of methanol. In regard to this ICG, 34.7 mg of 20-fold (molar ratio) nicotinamide was dissolved in 4 mL of methanol. These both methanol solutions were mixed together and stirred for 15 minutes, thereafter methanol was distilled away using an evaporator. 4 mL of chloroform was added to this substance, and stirred. Further, ultrasonic irradiation was performed for 30 seconds using an ultrasonic dispersion apparatus. Filtration was performed using a filter with a pore diameter of 0.45 micrometer, to collect dissolved components. The collected sample is defined as A-1.

### Example A-2, Comparative Example A-3

The same procedure as in Example A-1 was carried out except using benzyl nicotinate and pyridine, instead of using nicotinamide in A-1, to obtain A-2 and A-3.

### Comparative Example A-0

The same procedure as in Example A-1 was carried out except not using nicotinamide in Example A-1, to obtain A-0 that is a comparative control.

### Comparative Example A-4

The same procedure as in Example A-1 was carried out except using tetrabutylammonium iodide used in Journal of Biomedical Optics 131, 014025, 2008, instead of using nicotinamide, to obtain A-4, that is a comparative control.

### Examples B-1, B-2

The same procedure as in Examples A-1 and A-2 was carried out except using dichloromethane, instead of using chloroform in Examples A-1 and A-2, to obtain B-1 and B-2.

### Comparative Example B-0

The same procedure as in Example B-1 was carried out except not using nicotinamide in Example B-1, to obtain B-0, that is a comparative control.

### Comparative Example B-5

The same procedure as in Comparative Example B-0 was carried out except using a mixed solution of dichloromethane:methanol 3:1, instead of using dichloromethane in Comparative Example B-0, to obtain B-5, that is a comparative control.

### Comparative Example B-6

The same procedure as in Comparative Example B-0 was carried out except using a mixed solution of dichloromethane:methanol 1:1, instead of using dichloromethane in Comparative Example B-0, to obtain B-6, that is a comparative control.

### Comparative Example B-7

The same procedure as in Comparative Example B-0 was carried out except using a mixed solution of dichloromethane:methanol 1:3, instead of using dichloromethane in Comparative Example B-0, to obtain B-7, that is a comparative control.

### Examples C-1, C-2, C-3

The same procedure as in Example A-1 was carried out except using fursultiamine, prosultiamine and tiamine disulfide, instead of using nicotinamide in A-1, to obtain C-1, C-2 and C-3, respectively.

(Evaluation of Solubility)

The A-0 to A-4 solutions collected from the Examples and Comparative Examples were diluted 1000-fold, and UV-VIS-NIR measurement was carried out according to an ordinary method. Absorbencies at 550 nm to 950 nm were added up, and normalized with the value obtained in A-0 as 1. FIG. 1 compares the normalized values.

As compared to the control composition (A-0) including the dye and the solvent, very good solubility was shown, like 3.2 times in the case of using nicotinamide (A-1) and 2.1 times in the case of using benzyl nicotinate (A-2).

In addition, even compared to the case of (A-4) using a tetrabutylammonium salt used in Journal of Biomedical Optics 131, 014025, 2008, very good solubility was shown, like 3.2 times in the case of using nicotinamide (A-1) and 2.1 times in the case of using benzyl nicotinate (A-2).

Also, effectiveness was not seen in the case of using pyridine (A-3).

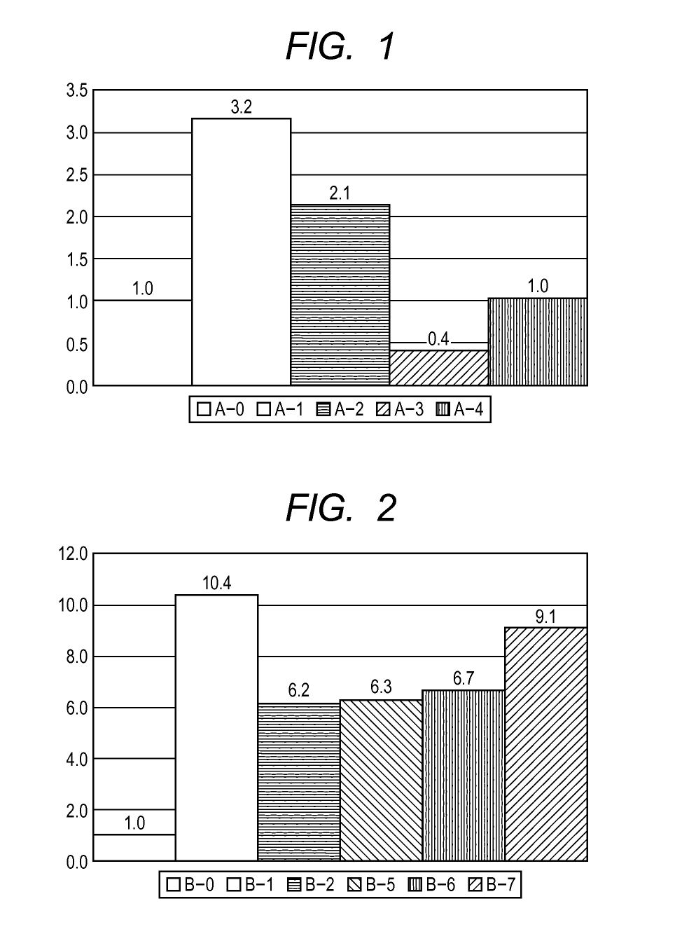

The B-0 to B-7 solutions collected from the Examples and Comparative Examples were diluted 1000-fold, and UV-VIS-NIR measurement was carried out according to an ordinary method. Absorbencies at 550 nm to 950 nm were added up, and normalized with the value obtained in Comparative Example B-0 as 1. FIG. 2 compares the normalized values.

As compared to the control composition (B-0) including the dye and the solvent, very good solubility was shown, like 10.4 times in the case of using nicotinamide (B-1) and 6.2 times in the case of using benzyl nicotinate (B-2).

In addition, as compared to B-0, very good solubility was shown, like 6.3 times to 9.1 times in the case of using methanol (B-5, B-6, B-7) depending on the amount of methanol added.

The C-1 to C-3 and A-0 solutions collected from the Examples and Comparative Examples were diluted 1000-fold, and UV-VIS-NIR measurement was carried out according to an ordinary method. Absorbencies at 550 nm to 950 nm were added up, and normalized with the value obtained in A-0 as 1 for comparison.

As compared to the control composition (A-0) including the dye and the solvent, very good solubility was shown, like 1.4 times in the case of using fursultiamine (C-1), 1.6 times in the case of using prosultiamine (C-2), and 1.9 times in the case of using tiamine disulfide (C-3).

(Example WB Evaluation of Transferability from Hydrophobic Solvent to Water)

To 0.5 mL of the B-0, B-1, B-2, B-5, B-6 and B-7 solutions prepared in the Examples and Comparative Examples was added 1.5 mL of ultra pure water, and the mixtures were shaken for 3 seconds. The mixtures were allowed to stand for 5 minutes, thereafter the aqueous layers were collected, and these samples was defined as WB-0, WB-1, WB-2, WB-5, WB-6 and WB-7, respectively.

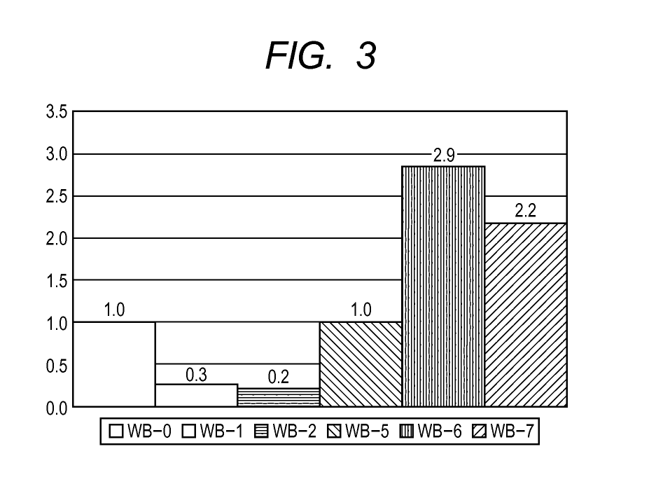

UV-VIS-NIR measurement was carried out according to an ordinary method. Absorbencies at 550 nm to 950 nm were added up and normalized the amount of ICG dissolved in the compositions WB-0, WB-1, WB-2, WB-5, WB-6 and WB-7, to calculate the amount transferred to water in the same amount of ICG. The values were normalized with the amount transferred in sample B-0 as 1. FIG. 3 compares the normalized values.

When methanol was used (WB-5, WB-6, WB-7), the amount transferred from the hydrophobic solvent to water showed large transferability like 1.0 time to 2.9 times as compared to the amount transferred in WB-0.

On the other hand, when nicotinamide (WB-1) and benzyl nicotinate (WB-2) were used, the amount transferred from the hydrophobic solvent to water both showed small values like 0.3 times and 0.2 times as compared to the amount transferred in WB-0.

Outline of each sample used in Examples A, B and WB are shown in Table 1.

TABLE 1

| | | Nicotinic Acid Derivative | | |
|---|---|---|---|---|
| | Hydrophilic | or Comparative Control | | |
| Sample | Dye | Substance | Solvent | Remark |
| A-0 | ICG | | Chloroform | |
| A-1 | ICG | Nicotinamide | Chloroform | |
| A-2 | ICG | Benzyl nicotinate | Chloroform | |
| A-3 | ICG | Pyridine | Chloroform | |
| A-4 | ICG | Tetrabutylammonium Salt | Chloroform | |
| B-0 | ICG | | Dichloromethane | |
| B-1 | ICG | Nicotinamide | Dichloromethane | |
| B-2 | ICG | Benzyl nicotinate | Dichloromethane | |
| B-5 | ICG | | Dichloromethane:Methanol | |
| | | | (3:1) | |
| B-6 | ICG | | Dichloromethane:Methanol | |
| | | | (1:1) | |
| B-7 | ICG | | Dichloromethane:Methanol | |
| | | | (1:3) | |
| C-1 | ICG | Fursultiamine | Chloroform | |
| C-2 | ICG | Prosultiamine | Chloroform | |
| C-3 | ICG | Tiamine Disulfide | Chloroform | |
| WB-0 | ICG | | Dichloromethane | Transferred to |
| | | | | Aqueous Layer |
| WB-1 | ICG | Nicotinamide | Dichloromethane | Transferred to |
| | | | | Aqueous Layer |
| WB-2 | ICG | Benzyl nicotinate | Dichloromethane | Transferred to |
| | | | | Aqueous Layer |
| WB-5 | ICG | | Dichloromethane:Methanol | Transferred to |
| | | | (3:1) | Aqueous Layer |
| WB-6 | ICG | | Dichloromethane:Methanol | Transferred to |
| | | | (1:1) | Aqueous Layer |
| WB-7 | ICG | | Dichloromethane:Methanol | Transferred to |
| | | | (1:3) | Aqueous Layer |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-187500, filed Aug. 24, 2010, which is hereby incorporated by reference herein in its entirety.

What is claimed is:

1. A composition comprising a hydrophilic dye comprising a sulfonate group and a hydrophobic solvent, wherein the composition comprises at least one of a nicotinic acid derivative and a tiamine derivative.

2. The composition according to claim 1, wherein the hydrophilic dye comprising a sulfonate group is indocyanine green.

3. The composition according to claim 1, wherein the nicotinic acid derivative is represented by formula (I):
A-Z (I)
(in the formula (I), A represents one of formulae (a1), (a2) and (a3) below, and
Z represents one of formulae (z1), (z2) and (z3) below) 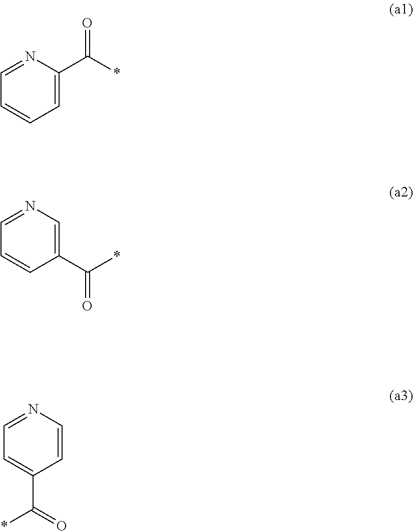
(in the formulae (a1), (a2) and (a3), * represents a bonding hand bound to Z of formula (I)) 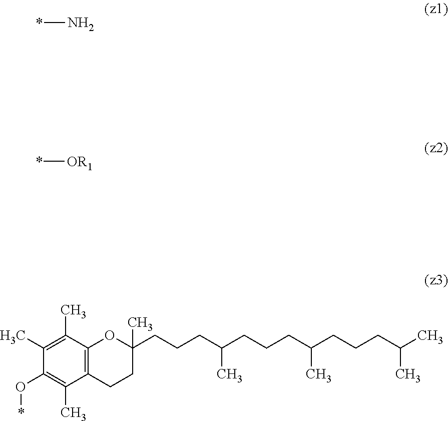
(in the formulae (z1), (z2) and (z3), * represents a bonding hand bound to A of formula (I),
in the formula (z2), R₁ is one of a hydrogen atom, a substituted or unsubstituted alkyl group having 1 to 10 carbon atoms and a benzyl group, and
the substituent is one of a halogen atom, an alkyl group having 1 to 5 carbon atoms, a hydroxyl group and an amino acid).

4. The composition according to claim 1, wherein the tiamine derivative is represented by formula (II):
X—B—Y (II)
(in the formula (II), B represents the formula (b) below, X represents one of formulae (x1), (x2), (x3) and (x4) below, and
Y represents one of formulae (y1) and (y2) below) 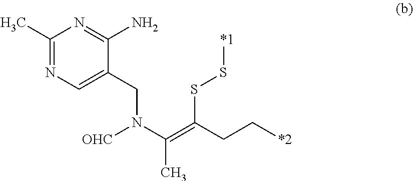
(in the formula (b), *1 represents a bonding hand bound to X of the formula (II), *2 represents a bonding hand bound to Y of the formula (II)) 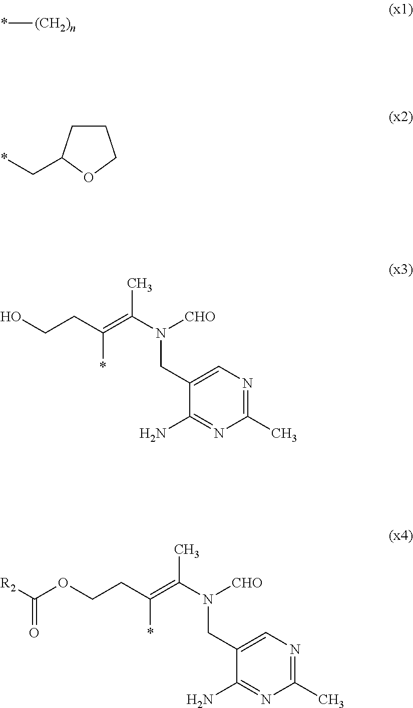
(in the formulae (x1), (x2), (x3) and (x4), * represents a bonding hand bound to B of formula (II),
in the formula (x1), n is an integer selected from 1 to 10, the formula (x1) may be substituted by a halogen atom, an alkyl group having 1 to 5 carbon atoms, a hydroxyl group and an amino acid,
in the formula (x4), R₂ is one of a substituted or unsubstituted alkyl group having 1 to 10 carbon atoms and a substituted or unsubstituted benzene) 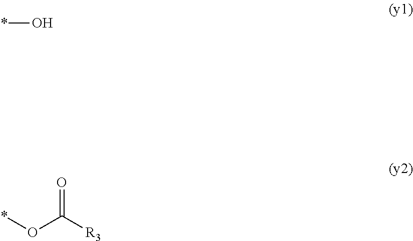
(in the formulae (y1) and (y2), * represents a bonding hand bound to B of formula (II),
in the formula (y2), R₃ is one of a substituted or unsubstituted alkyl group having 1 to 10 carbon atoms and a substituted or unsubstituted benzene).

5. The composition according to claim 1, wherein the nicotinic acid derivative is at least one of nicotinamide and benzyl nicotinate.

6. The composition according to claim 1, wherein the tiamine derivative is at least one of fursultiamine, prosultiamine and tiamine disulfide.

7. The composition according to claim 1, wherein the hydrophobic solvent is at least one of chloroform and dichloromethane.

8. A method for producing a contrast agent for optical imaging, wherein the method comprises the steps of:
preparing a composition comprising a hydrophilic dye comprising a sulfonate group, a hydrophobic solvent, at least one of a nicotinic acid derivative and a tiamine derivative, and one of a polymerizable monomer and a prepolymer,
mixing the composition with water, and
polymerizing one of the polymerizable monomer and the prepolymer after the mixing step.
